(19) United States
(12) Patent Application Publication
(10) Pub. No.: US 2026/0257016 A1
(43) Pub. Date: Sep. 3, 2026

(54) AUTOINJECTOR KITS

(71) Applicant: NOVARTIS AG, Basel (CH)

(72) Inventors: Neil CAMMISH, Lancashire (GB); Mark HORLOCK, Cheshire (GB); Claudio ROSSI, Basel (CH)

(21) Appl. No.: 18/873,005

(22) PCT Filed: Jun. 9, 2023
(86) PCT No.: PCT/IB2023/055983

§ 371 Date: Dec. 9, 2024

Related U.S. Application Data
(60) Provisional application No. 63/351,009, filed on Jun. 10, 2022.

(51) Int. Cl. A61M 5/20; A61M 5/315; A61M 5/32

(52) U.S. Cl. CPC A61M 5/2033; A61M 5/31591; A61M 5/3202

(57) ABSTRACT

An autoinjector kit, the autoinjector kit comprising an autoinjector device comprising: a longitudinal housing extending along a longitudinal axis and having an open proximal end to contact a trigger site, a distal end opposite to the proximal end and a hollow interior; a barrel body mounted inside the housing; a stopper slidably mounted within the barrel body and movable from a distal position to a proximal position by a plunger rod; a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position; a trigger mechanism operable to release the plunger rod and initiate a transition of the drive mechanism from the primed state to the fired state to thereby cause the plunger rod to drive the stopper from the distal position to the proximal position; an indicator component which, when triggered, moves distally from an initial position to an indication position to indicate a state of the autoinjector device; wherein the autoinjector kit comprises an indicator reset and the housing of the autoinjector device comprises a reset access opening in the distal end of the longitudinal housing, the indicator reset comprising a base and a reset projection, the reset projection insertable through the reset access opening to contact the indicator component and force the indicator component into the initial position.

The present invention is directed to autoinjector kits comprising autoinjector devices, in particular those comprising training or demonstration autoinjector devices which may be intended to simulate at least some of the operations of an automatic drug delivery device. The invention extends to autoinjector device and to methods of using such devices.

Autoinjector devices, also sometimes called automatic drug delivery devices, are known examples of medical devices. Such autoinjector devices are configured for dispensing a fluid product such as a medicament to a patient. The term autoinjector device, or autoinjector, is used herein to describe not only an automatic drug delivery device described above, but also a training or demonstration device which may be intended to simulate at least some of the functions of such an automatic drug delivery device.

Examples of such autoinjector devices for drug delivery are described, for example, in WO 2019/224782, WO 2019/224783, WO 2019/224784 and WO 2019/224785, the contents of which are incorporated herein by reference.

In general, such autoinjector devices provide reliable functioning and may be used accurately even by non-experienced persons. Nevertheless, there is still a need to train patients or caregivers in the use of such devices and this can be a potentially time-consuming process and may involve the demonstration of operation of a device a plurality of times. Some autoinjector devices may comprise electronic components that allow capturing of training attempts and may feedback to the user and/or care provider. Such devices provide the potential for training to be administered and/or monitored remotely rather than face to face.

The invention provides an autoinjector kit, the autoinjector kit comprising an autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position;
a trigger mechanism operable to release the plunger rod and initiate a transition of the drive mechanism from the primed state to the fired state to thereby cause the plunger rod to drive the stopper from the distal position to the proximal position;
an indicator component which, when triggered, moves distally from an initial position to an indication position to indicate a state of the autoinjector device; wherein
the autoinjector kit comprises an indicator reset and the housing of the autoinjector device comprises a reset access opening in the distal end of the longitudinal housing, the indicator reset comprising a base and a reset projection, the reset projection insertable through the reset access opening to contact the indicator component and force the indicator component into the initial position.

The indicator component may be any suitable element which, when triggered, moves distally from an initial position to an indication position to indicate a state of the autoinjector device. In the indication position the indicator component may be visible to a user, for example through a window, to indicate a state of the autoinjector device. On arriving in the indication position the indicator component may provide an audible and/or tactile indication to indicate a state of the autoinjector device. The indicator component may provide a combination of visible, audible and/or tactile indications regarding a state of the autoinjector device. The state being indicated may be that a device operation has finished, for example a liquid dose has been delivered, or the stopper has reached a predetermined, for example the proximal position. The indicator element may stop at the indicator position or may pass through the indicator position.

The provision of an indicator reset, which may be referred to as an indicator reset element or an indicator reset device, the projection of which is able to pass through the reset access opening of the housing to contact the indicator component allows a user to move the indicator component back to the initial position. By allowing a user to move the indicator component back to its initial position a resetting of the autoinjector device is made possible without a need to dismantle some, or all, of the components. When the indicator component is moved back to the initial position it may be retained in that initial position until triggered by a subsequent actuation of the autoinjector device.

The reset access opening may have any shape that permits access to the indicator component for the projection of the indicator reset. The reset access opening may be a hole in a distal end of the longitudinal housing which may allow a rod-shaped projection to pass therethrough and move the indicator component. The reset access opening may be an elongate slot extending from the distal end of the housing towards the proximal end, and the reset projection may comprise an elongate wall. Such an arrangement of slot and wall has been found to be capable of providing a stronger and more reliable mechanical interaction between the indicator component and the projection as deformation of the projection can be more easily controlled, reduced or avoided.

The indicator reset may comprise a case into which the housing can be fitted. The case may enclose some, or substantially all, of the longitudinal housing. If the longitudinal housing may be fitted with a cap, for example to cover the open proximal end, then the case may also enclose some or all of the cap.

When the housing is fitted into the case the housing may be releasably secured therein, for example by one or more releasable clips. The releasable clips may act upon the housing, on a cap coupled to the housing, or on any other part of the autoinjector device or component coupled thereto. The clips retain the housing by resisting a removal force which would act to remove the housing from the case. The releasable clips may be deformable such that the clips are released if a removal force above a threshold is applied. This may allow a user to simply pull the housing from the case, for example by pulling on the housing, or on a cap coupled thereto. In other examples the releasable clips may prevent removal of the housing from the case until the clips are released by a user manipulating a release member, for example pressing a button, sliding a release catch, moving a release lever or other manipulation.

The case may be configured such that as the housing is fitted into the case, the reset projection of the case extends through the reset access opening of the housing and forces the indicator component into the initial position. This may provide a convenient way for a user to reset the indicator component as they simply return the used autoinjector to the associated case. The case may provide a convenient way for a user to store the autoinjector device.

The shape of the case and/or the housing may be configured such that the housing can only enter the case is one rotational orientation about the longitudinal axis. In this way the user may be able to insert the housing into the case in a single orientation in which the reset projection of the indicator reset may be aligned with the reset access opening.

The indicator reset and/or the case may include a removal indicator. When the indicator reset is fully inserted, or the autoinjector device is inserted into the case the removal indicator may interact with a part of the autoinjector such that removal of the indicator reset, or removal of the autoinjector device from the case, results in a change in state of a component of the autoinjector device thereby allowing removal of indicator reset or removal of the autoinjector device from the case to be detected, for example by an electronic module of the autoinjector. Facilitating detection of the removal of the indicator reset, or removal of the autoinjector device from the case by an electronic module may facilitate turning on, waking up and/or initialising the electronic module prior to use.

An electronic module of the autoinjector device may include an electrical power source, such as a capacitor or battery, and the electrical power source may be rechargeable. The electrical power source can be removed from the autoinjector device for charging, or may be recharged in the autoinjector housing.

Recharging the electrical power source in the autoinjector housing may be through wired charging, for example plugging a power source into the autoinjector device, or into something into which the autoinjector is placed, for example a case. In some examples a case for the autoinjector may comprise a power source such as a battery or capacitor for recharging the electrical power source of the autoinjector device. The case and autoinjector device may be configured such that, when the autoinjector device is placed in the case, a connection, wired or wireless, is made so that the electrical power source of the autoinjector is charged.

In some examples the indicator reset may include a portion that contacts and resets the indicator component, and a further portion that contacts and moves another component of the autoinjector, for example a trigger component. Removal of the indicator reset may cause the other component to move to its starting position and such movement may be detected by the electronic module, for example by a change in state of a contact switch, movement of a magnetic or other detectable element or by a proximity sensor. The indicator reset may comprise, for example, a stepped rod or wall, where a first step contacts and resets the indicator component, and a further step contacts and moves another component of the autoinjector.

In some examples the indicator component may be retained by the indicator reset and/or case in a position which is proximal of its initial position such that removal of the indicator reset, or removal of the autoinjector device from the case, results in a distal movement of the indicator component to its initial position where it may be retained until triggered.

In some examples the case may comprise a detectable portion, for example a detectable portion which can be detected without contact, for example a magnetic or metallic portion, or a detectable portion which can be detected through contact, for example a projection, ridge or catch. When the autoinjector device is fully inserted in the case the detectable portion may be detectable by an electronic module of the autoinjector, for example by a sensor or switch. Removal of the autoinjector device from the case moves the detectable portion of the case away from the electronic module meaning that the electronic module can detect removal of the autoinjector device from the case.

The longitudinal housing of the autoinjector device may be any suitable shape and may be elongate. The longitudinal housing has a hollow interior and extends along the longitudinal axis from an open proximal end to a distal end opposite the proximal end. The longitudinal housing may comprise a plurality of components.

The open proximal end is to contact a trigger site, in some examples a needle guard component of the longitudinal housing may provide the open proximal end and may contact the trigger site. Forcing the longitudinal housing against the trigger site may cause movement of the needle guard relative to a shell component of the longitudinal housing, for example forcing the needle guard moving into the shell component, may trigger actuation of the autoinjector device. The trigger site may be a part of a body of a patient, for example a thigh or stomach, or may be a test pad which may simulate a body part.

The open proximal end allows access therethrough into the hollow interior which houses components of the autoinjector device, including a barrel body which is mounted inside the longitudinal housing. The barrel body may be, for example, a syringe barrel, which for a drug delivery device may contain a liquid, for example a liquid medicament, to be delivered to a patient. In a demonstration, or training, device the barrel body may be a replica of a syringe barrel, for example a component intended to replicate the ‘look’ and/or ‘feel’ of those parts of a syringe barrel that can be seen or felt when using an autoinjector.

Within the barrel body a stopper is slidably mounted and can slide from a distal position to a proximal position. In the proximal position the stopper is closer to the open proximal end of the longitudinal housing than it is when in the distal position. In a drug delivery device the stopper may define a variable volume chamber with the barrel body. When the stopper is moved from the distal position to the proximal position within the barrel body the volume of the variable volume chamber is reduced and any liquid contained therein may be expelled from the variable volume chamber, for example through a needle which may be coupled to, or integrally formed with, a proximal end of the barrel body.

The autoinjector device includes a drive mechanism which is able to drive a plunger rod to cause movement of the stopper from the distal position to the proximal position. To provide a driving force to drive mechanism may comprise any suitable energy storage and may include mechanical springs, for example coil or leaf springs, or gas springs, for example damped gas springs, or combinations thereof.

In the primed state the plunger rod is held, directly or indirectly, against a driving force in a retracted position between the distal end and the stopper in the distal position. In the retracted position the plunger rod may be spaced from the stopper in the distal position. In the primed state any energy storage may be in an energised state, for example mechanical springs compressed, or gas springs pressurised.

The speed of movement of a gas spring can be predetermined to control the speed of movement of the plunger rod and stopper during use. This may facilitate the creation of a demonstration or training device which more accurately replicates the action of a medication delivery device, or a drug delivery device delivers the drug in a predetermined time.

In the fired state the plunger rod extends through at least a portion of the barrel body and is in contact with the stopper when the stopper is in the proximal position. At least some of the energy stored in the primed position has been used to drive the plunger rod toward the open proximal end of the longitudinal housing.

The autoinjector device comprises a trigger mechanism which is operable to release the plunger rod. This initiates a transition of the drive mechanism from the primed state to the fired state to thereby cause the plunger rod to drive the stopper from the distal position to the proximal position. As suggested above the trigger mechanism may be operated by relative movement between parts of the longitudinal housing, for example movement of a needle guard relative to a shell. The trigger mechanism may be operated by a user operated button, switch, catch or lever, which may be, for example, mounted on the distal end or side of the longitudinal housing.

The stopper may comprise a stopper opening extending through the stopper along the longitudinal axis. The autoinjector kit may further comprise a reset rod. The reset rod may have a first end that is insertable through the open proximal end of the housing to contact the plunger rod through the stopper opening and force the plunger rod into the retracted position and thereby transition the drive mechanism to its primed state. The reset rod may have a second end opposite the first that remains outside the longitudinal housing as the plunger rod is moved to the retracted position. This provides a simple reset mechanism for the plunger rod and drive mechanism an autoinjector device, particularly for a demonstration or training device. When the plunger rod and drive mechanism are moved back to their initial conditions they may be retained until triggered by a subsequent actuation of the autoinjector device.

The reset rod may comprise a protrusion which is offset from the first end along the reset rod. The protrusion may be sized so that it cannot pass through the stopper opening and so may contact the stopper. The protrusion may provide a bearing surface to contact the stopper. The protrusion may contact the stopper and move the stopper to the distal position as the plunger rod is moved into the retracted position.

A portion of protrusion may extend substantially perpendicular to the longitudinal axis. The protrusion may be any suitable shape, for example a swollen portion of the reset rod. The protrusion may comprise a circumferential shoulder. The protrusion may be integrally formed with the reset rod, or may be a separate element carried by, or attached to, the reset rod. A circumferential shoulder may provide a continuous bearing surface, for example a flat bearing surface, which may provide greater control over the position along the reset rod at which the protrusion contacts the stopper.

The offset between the first end and the protrusion may be greater than an extent of the stopper along the longitudinal axis. This may create a space between the plunger rod in the retracted position and the stopper in the distal position.

In some examples of a drug delivery device there may be a gap between the plunger rod and the stopper when the device is ready to be used. When triggered, the plunger rod may move rapidly towards and impact the stopper creating an audible and tactile feedback. For a demonstration or training device, recreating such audible and tactile feedback is beneficial so providing a simple mechanical way in which, while resetting the plunger rod, the stopper is also reset and the original gap between the stopper and plunger rod is recreated. In this way subsequent actuations can recreate any audible and tactile feedback provided by the original gap between the plunger rod and the stopper.

The second end of the reset rod may comprise a cap mountable to the proximal end of the housing. In this way, recapping the device after use can reset the plunger rod and stopper to their initial positions. The cap may be rotated to remove the cap from the housing. Providing a flat bearing surface for the protrusion may reduce damage to the stopper if the reset rod is rotated relative to the stopper, for example if the cap is rotated to remove it from the housing.

The autoinjector device may comprise a guide track to control rotation of the plunger rod and/or stopper about the longitudinal axis during operation. Controlling rotation of the plunger rod during operation may make the reset operation more reliable, or simpler, as circumferentially distributed features, for example catches for retaining the plunger rod in the retracted position may be automatically aligned for reengagement.

In some examples the plunger rod may be secured to the stopper. The plunger rod may be secured to the stopper so that rotation of the plunger rod relative to the stopper is restricted or prevented. The stopper and/or the plunger may comprise an axial ridge or a circumferential projection on an outer surface which may engage with an axial slot in the barrel body to control rotation of the plunger rod and/or stopper about the longitudinal axis during operation. The axial slot in the barrel body may provide the guide track which is used to control rotation of the plunger rod and/or stopper via the stopper.

As noted above, the autoinjector device may include a reset rod which is insertable through the open proximal end of the housing to apply a reset force to the plunger rod to move it into the retracted position and thereby transition the drive mechanism to its primed state. In some examples the stopper may comprise a stopper opening extending through the stopper along the longitudinal axis so that the reset rod can pass therethrough and contact the plunger rod. In other examples the stopper may comprise no such stopper opening and the reset rod may contact the stopper and apply a force thereto to thereby apply a force to the plunger rod.

The maximum reset force required to move the plunger rod into the retracted position may be below 20 N, below 10 N or below 5 N as this facilitates resetting of the device. This may be particularly appropriate for a demonstration, or training device, in which the drive mechanism does not act on a stopper to drive liquid from a syringe into a patient. A comparatively low reset force may have other advantages for resetting the device as releasable catches and detents can be more flexible than if they were to restrain a stronger force such as restraining a drive spring that might be required to drive a plunger to dispense a volume of liquid medicament through a needle within an acceptable time.

An internal dimension of the barrel body perpendicular to the longitudinal axis may increase towards the proximal end of the autoinjector device such that compression of the stopper is reduced as the stopper is driven by the plunger rod towards the proximal end. The stopper may be initially compressed by a first amount when in the distal position and this amount may reduce as the stopper moves towards the proximal position.

The reduction in compression of the stopper may reduce the force required to cause the stopper to slide within the barrel body. If the plunger is being driven by a spring this can lead to a more consistent stopper movement speed. The driving force of a spring may be reduced as it expands and the resistive force provided by friction of the stopper with the barrel body may be adjusted, for example reduced, and this may compensate. This allows the speed of movement of the stopper to be controlled. In some examples the stopper may move at a substantially constant speed. This may be particularly relevant for stoppers made from elastomeric or compressible rubber-like materials that have a relatively high Poisson's ratio. For example thermoplastic elastomers and rubbers may have a Poisson's ratio of 0.45-0.50.

The barrel body may be substantially circular in cross section and the internal dimension may be an internal diameter of the barrel body. The rate of reduction of the internal dimension may be substantially constant resulting a draft angle relative to the longitudinal axis of between 0.05° and 0.3°.

The stopper may be made in any suitable shape and may include a one or more known features such as ribs and valleys. The stopper may be made of any suitable material, for example be made of any suitable material or materials, for example a plastic, elastomer, rubber or silicone or combination thereof. The material may be selected to provide a desired resistance to deformation and/or a desired coefficient of friction. The resistance to deformation and/or a desired coefficient of friction influence the resistance to movement of the stopper relative to the barrel body, for example when the stopper is deformed to fit into the barrel body.

The stopper and/or barrel body may have a coating, for example an oil or grease applied to a contact surface to adjust the resistance to movement of the stopper relative to the barrel body when the stopper is moved within the barrel body. The coating may be a synthetic oily liquid or emulsion such as a polydimethylsilicone liquid which may be applied on the stopper and/or the barrel body. The coating may be a solid film layer like PTFE applied, for example, to the stopper.

The invention also provides an autoinjector kit, the autoinjector kit comprising an autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position; and
a trigger mechanism operable to release the plunger rod and initiate a transition of the drive mechanism from the primed state to the fired state to thereby cause the plunger rod to drive the stopper from the distal position to the proximal position; wherein
the stopper comprises a stopper opening extending through the stopper along the longitudinal axis and the autoinjector kit further comprises a reset rod, the reset rod having a first end that is insertable through the open proximal end of the housing to contact the plunger rod through the stopper opening and force the plunger rod into the retracted position and thereby transition the drive mechanism to its primed state, the reset rod having a second end opposite the first that remains outside the longitudinal housing as the plunger rod is moved to the retracted position.

As set out above, providing a stopper opening extending through the stopper provides a convenient way to reset the position of both the stopper and plunger rod.

The invention further provides an autoinjector device, the autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position; and
a trigger mechanism operable to release the plunger rod and initiate a transition of the drive mechanism from the primed state to the fired state to thereby cause the plunger rod to drive the stopper from the distal position to the proximal position; wherein
the autoinjector device comprises a guide track to control rotation of the plunger rod about the longitudinal axis during operation.

As set out above, controlling rotation of the plunger rod may facilitate resetting of the plunger rod.

The invention further provides an autoinjector kit, the autoinjector kit comprising an autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position;
a trigger mechanism operable to release the plunger rod and initiate a transition of the drive mechanism from the primed state to the fired state to thereby cause the plunger rod to drive the stopper from the distal position to the proximal position; and
the autoinjector kit comprising a reset rod, the reset rod having a first end that is insertable through the open proximal end of the housing to apply a reset force to the plunger rod to move it into the retracted position and thereby transition the drive mechanism to its primed state, the rod having a second end opposite the first that remains outside the longitudinal housing as the plunger rod is moved to the retracted position; wherein
the maximum reset force required to move the plunger rod into the retracted position is below 10 N.

As set out above, controlling the reset force to be below a predetermined threshold may make it easier for a user to reset the device. The force required to reset parts of the autoinjector device, which may include resetting of the indicator, the plunger rod and stopper, and the trigger, as well as attaching the cap may be in series (sequential) so as to reduce the peak forces required.

The invention also provides an autoinjector device, the autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position; and
a trigger mechanism operable to release the plunger rod and initiate a transition of the drive mechanism from the primed state to the fired state to thereby cause the plunger rod to drive the stopper from the distal position to the proximal position; wherein
an internal dimension of the barrel body perpendicular to the longitudinal axis increases towards the proximal end of the autoinjector device such that compression of the stopper is reduced as the stopper is driven by the plunger rod towards the proximal end.

As set out above, increasing an internal dimension of the barrel body in this way provides a way of controlling the speed of movement of the stopper when driven by the plunger rod.

The invention may also provide an autoinjector device suitable for use with any of the kits mentioned above.

The invention extends to a method of resetting an indicator component in an autoinjector device, the autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position;
a trigger mechanism operable to release the plunger rod and initiate a transition of the drive mechanism from the primed state to the fired state to thereby cause the plunger rod to drive the stopper from the distal position to the proximal position; and
an indicator component which, when triggered, moves distally from an initial position to an indication position to indicate a state of the autoinjector device; wherein the method comprises:
inserting a reset projection of an indicator reset though a reset access opening in the distal end of the longitudinal housing of the housing of the autoinjector device to contact the indicator component and using reset projection to force the indicator component into the initial position.

A user may hold the indicator reset by a base to facilitate insertion of the reset projection though the reset access opening. The base may comprise guides to facilitate alignment of the reset projection and reset access opening. The base may comprise a case for the autoinjector device as described above.

To reset the indicator component by the returning it to its initial position a user may align the reset access opening of the autoinjector with the reset projection and move the autoinjector device relative to the reset projection such that the reset projection enters the reset access opening and contacts the indicator component. Further relative movement between the autoinjector device and the reset projection may cause the reset projection to force the indicator component into the initial position.

The alignment and relative movement may be achieved, for example, by a user inserting the autoinjector device into a case which includes the reset projection and which aligns the autoinjector device with the reset projection. This provides a simple way of resetting the indicator component after operation of the device.

The invention may also include a method of resetting a plunger rod of an autoinjector device, the autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position; and
a trigger mechanism operable to release the plunger rod and initiate a transition of the drive mechanism from the primed state to the fired state to thereby cause the plunger rod to drive the stopper from the distal position to the proximal position; wherein the method comprises:
inserting a first end of a reset rod through the open proximal end of the housing to contact the plunger rod through a stopper opening, the stopper opening extending through the stopper along the longitudinal axis; and
using the reset rod to force the plunger rod into the retracted position and thereby transitioning the drive mechanism to its primed state, a second end of the reset rod opposite the first end remains outside the longitudinal housing as the plunger rod is moved to the retracted position.

A user may hold the reset by the second end to facilitate insertion of the reset rod through the open proximal end of the housing. The second end may comprise a cap for closing or covering the open proximal end of the housing of the autoinjector device as described above.

By inserting the first end of the reset rod through the open proximal end of the housing and using it to force the plunger into the retracted position the plunger rod and drive mechanism are reset as the plunger rod is returned to the retracted position and the drive mechanism to its primed state. If the second end of the reset rod comprises a cap the cap may engage with the housing to close or cover the open proximal end of the housing when the plunger reaches the retracted position.

Thus, returning the cap to the autoinjector device after use may comprise a convenient way to reset the plunger rod and drive mechanism after operation of the device.

An example sequence of use may comprise the following actions. A user may remove the autoinjector from a case and remove a cap from the autoinjector device to ready the autoinjector device for use. These actions could occur in any order, but in some examples the case may hinder removal of the cap until the autoinjector device is removed from the case.

The autoinjector device can then be actuated by pressing the open proximal end against a trigger site.

Once used, elements of the autoinjector can be reset as described above. This can be achieved by, for example, by a user inserting a first end of a reset rod into the open proximal end and/or inserting a reset projection of an indicator reset into a reset access opening. In some embodiments these reset actions can be achieved by returning the autoinjector device to the case and placing the cap back on the open proximal end. The case may comprise guides to facilitate alignment between the cap and the autoinjector device. Inserting the autoinjector device into the case may also automatically initiate charging of an electrical power source of the autoinjector device.

The invention will now be described by way of example only with reference to the following figures in which:

FIG. 1 shows an example of an autoinjector device prior to use;

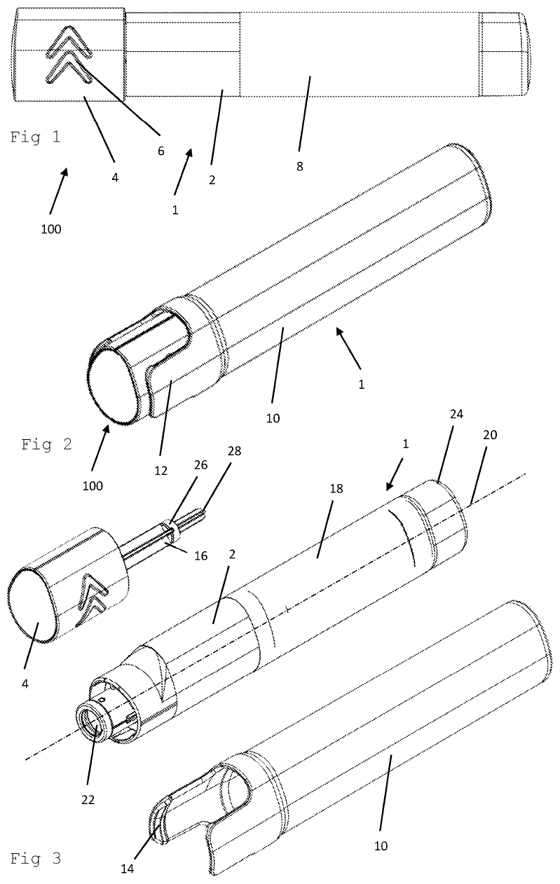

FIG. 2 shows an example of an autoinjector device in a case prior to use;

FIG. 3 shows an example of an autoinjector device removed from a case prior and with the cap removed ready for use;

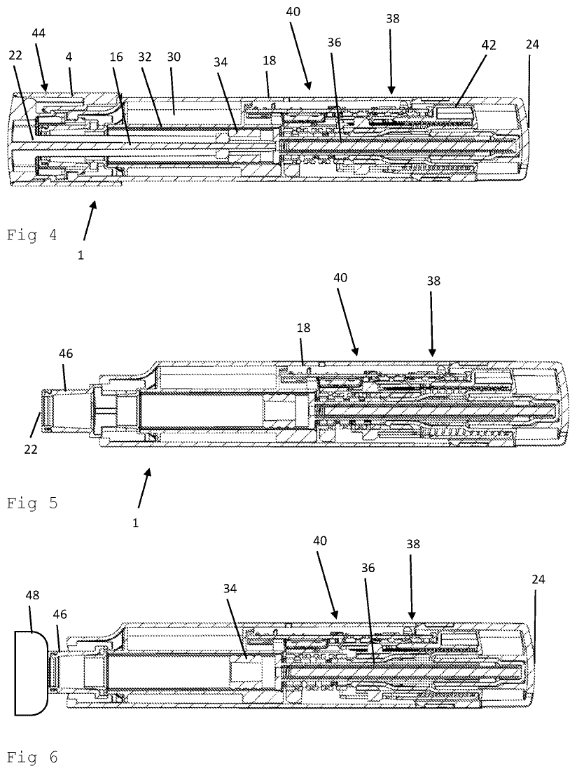

FIG. 4 shows a cross section through an example of an autoinjector device prior to use;

FIG. 5 shows a cross section through an example of an autoinjector device with the cap removed;

FIG. 6 shows a cross section through an example of an autoinjector device with a needle guard member of the housing pressed against a trigger site at the start of plunger rod movement;

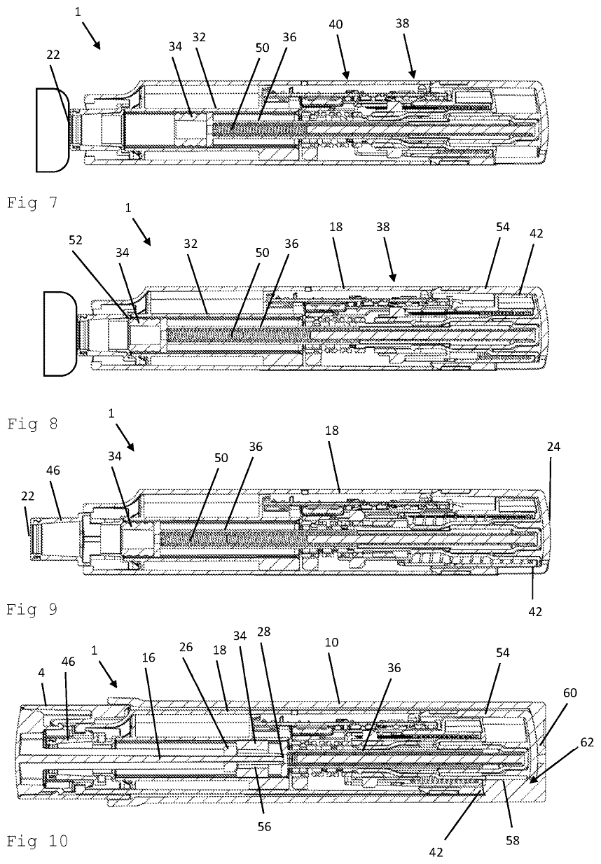

FIG. 7 shows a cross section through an example of an autoinjector device with a needle guard member of the housing pressed against a trigger site during operation plunger rod movement;

FIG. 8 shows a cross section through an example of an autoinjector device with a needle guard member of the housing pressed against a trigger site at the end of plunger rod movement;

FIG. 9 shows a cross section through an example of an autoinjector device following plunger rod movement with a needle guard member of the housing moved away from a trigger site;

FIG. 10 shows a cross section through an example of an autoinjector device having been placed into a case and recapped following use;

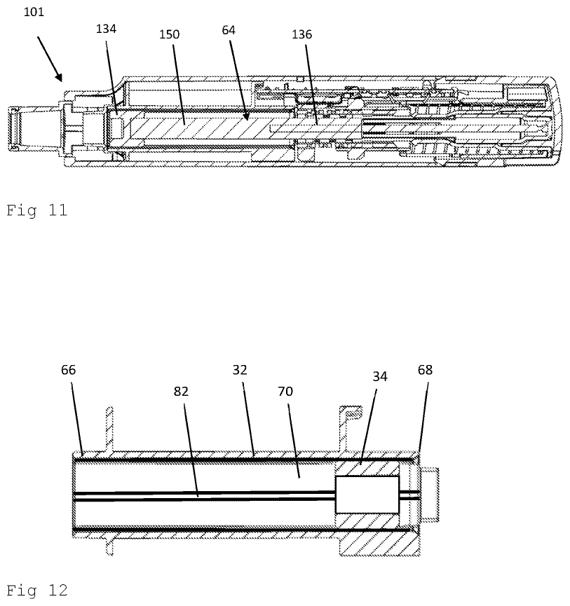

FIG. 11 shows a cross section through a different example of an autoinjector device with the cap removed;

FIG. 12 is a cross section through a barrel body of an autoinjector device;

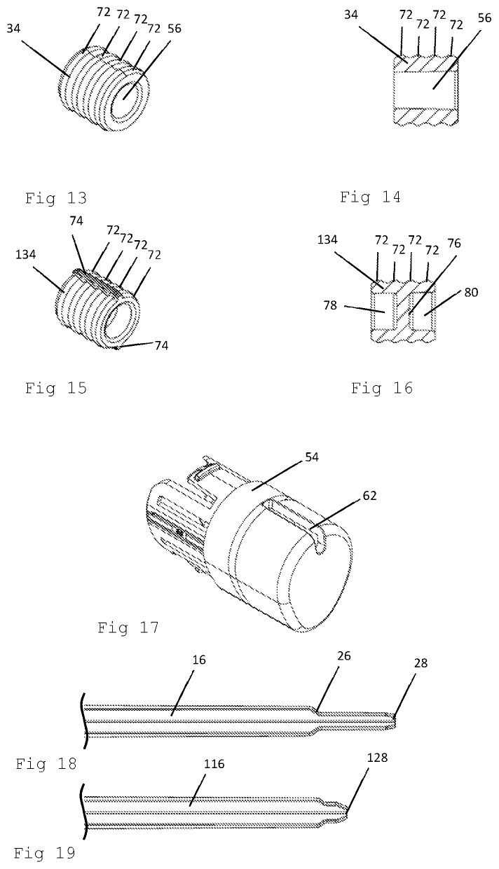

FIG. 13 is a view of an example stopper;

FIG. 14 is a cross section through the example stopper of FIG. 13;

FIG. 15 is a view of an example stopper;

FIG. 16 is a cross section through the example stopper of FIG. 15;

FIG. 17 is a view of an example end portion of a housing;

FIG. 18 is a view of an example of an end of a reset rod;

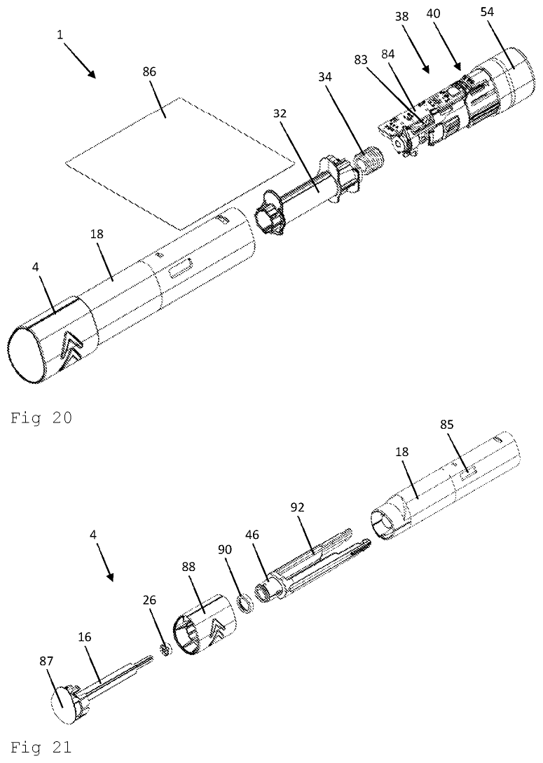

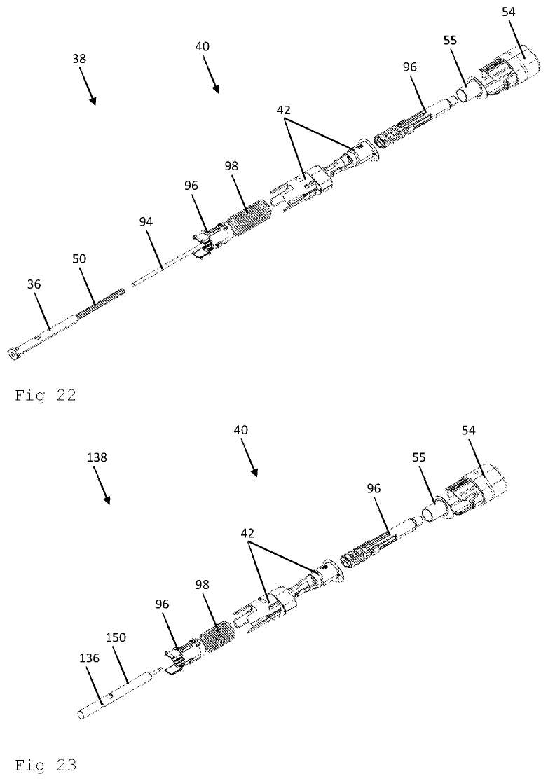

FIG. 19 is a view of a different example of an end of a reset rod;

FIG. 20 is an exploded view of an example autoinjector device;

FIG. 21 is an exploded view of a cap and proximal components of an example autoinjector device;

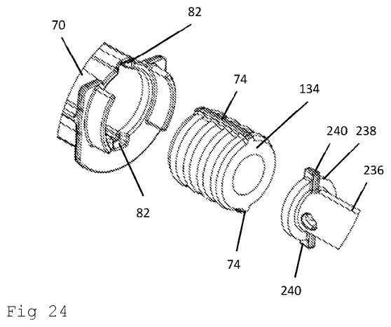

FIG. 22 is an exploded view of an example drive mechanism, plunger rod and housing endcap of an example autoinjector device;

FIG. 23 is an exploded view of another example drive mechanism, plunger rod and housing endcap of an example autoinjector device; and

FIG. 24 is an exploded view of an example of a barrel body, stopper and plunger rod.

FIG. 1 shows an example of an autoinjector kit 100 comprising an autoinjector device 1 and a cap 4. The autoinjector device 1 comprises a body 2. The cap 4 includes raised indicia 6 to provide information to a user to facilitate removal of the cap 4 from the body 2. In this example the body 2 is transparent or translucent so internal components can be seen by a user and a label 8 is wrapped around a portion of the body 2 to conceal some internal components. In other examples the body may be opaque entirely, or in portions which may define viewing windows, or viewing windows may be provided through the body 2.

FIG. 2 shows an example of an autoinjector kit 100, for example the autoinjector kit 100 of FIG. 1, comprising a case 10 in which the autoinjector device 1 can be stored prior to use. In this example the case 10 surrounds and contains the body 2 of the autoinjector 1 so that a user cannot touch the body 2. In other examples the case 10 may include cut-out portions.

The case 10 includes projections 12 which extend across the cap 4. The projections 12 include catches 14 (visible in FIG. 3) that engage with the raised indicia 6 to releasably retain the autoinjector 1 in the case 10. In other examples catches or other releasable retaining elements may be provided elsewhere on the case 10 or cap 4 so that the autoinjector device 1 can be releasably retained in the case 10.

FIG. 3 shows an exploded view of the autoinjector kit 100 of FIG. 2 with the cap 4, autoinjector device 1, and case 10 shown separately. The body 2 comprises a longitudinal housing 18 extending along a longitudinal axis 20 and having an open proximal end 22 to contact a trigger site (not shown in this Figure). The housing 18 extends along the longitudinal axis 20 from the open proximal end to a distal end 24 opposite to the open proximal end 22. The longitudinal housing 18 has a hollow interior.

A reset rod 16 extends from the cap 4 and can be inserted into the hollow interior of the housing through the open proximal end 22 of the housing 18. In this example the reset rod 16 includes an end portion 28 for engaging a plunger rod (not shown in this Figure) and a wider bearing element 26 for contacting a stopper (not shown in this Figure). In other examples the end portion 28 may contact the stopper directly.

FIG. 4 shows a cross section through an example of an autoinjector device 1 removed from the case 10, and with its cap 4 on, for example the autoinjector device 1 and cap 4 of FIG. 1. In this Figure the hollow interior 30 of the longitudinal housing 18 can be seen.

A barrel body 32 is mounted inside the housing 18 and a stopper 34 is slidably mounted within the barrel body 32. The stopper 34 is movable towards the open proximal end 22 from a distal position (as shown in this Figure) to a proximal position (as shown in FIG. 9) by a plunger rod 36. The distal position is closer to the distal end 24 of the housing 18 than the proximal position.

A drive mechanism 38 is connected to the plunger rod 36. In this Figure the drive mechanism 38 is shown in a primed state in which the plunger rod 36 is held against a driving force in a retracted position between the distal end 24 of the housing 18 and the stopper 34 in the distal position.

The drive mechanism 38 is movable to a fired state (as shown in FIG. 9) in which the plunger rod 36 extends through at least a portion of the barrel body 32 and contacts the stopper 34 in the proximal position.

The autoinjector 1 includes a trigger mechanism 40 operable to release the plunger rod 36 and initiate a transition of the drive mechanism 38 from the primed state to the fired state to thereby cause the plunger rod 36 to drive the stopper 34 from the distal position to the proximal position.

The autoinjector 1 includes an indicator component 42 which, when triggered, moves distally from an initial position (as shown in this Figure) to an indication position (as shown in FIG. 9) to indicate a state of the autoinjector device 1.

FIG. 5 shows a cross section through an example of an autoinjector device 1 with the cap 4 removed. The removal of the cap 4 allows a needle guard 46 of the housing 18 to extend along the longitudinal axis away from the drive mechanism 38. The needle guard 46 is coupled to the trigger mechanism 40 as will be described in outline below. A detailed example of a trigger mechanism 40 and associated drive mechanism 38 suitable for use in an autoinjector can be found in, for example, WO 2019/224782, WO 2019/224783, WO 2019/224784 and WO 2019/224785.

FIG. 6 shows a cross section through an example of an autoinjector device 1 with the needle guard 46 pressed against a trigger site 48. In this example the trigger site 48 is a pad which may replicate the texture of a human tissue into which an injection may be made.

The pressing of the needle guard 46 into the trigger site causes the needle guard 46 to move towards the distal end 24 of the housing and activate the trigger mechanism 40, releasing the plunger rod 36 and initiate a transition of the drive mechanism 38 from the primed state to the fired state to thereby cause the plunger rod 36 to drive the stopper 34 from the distal position to the proximal position.

FIG. 7 shows a cross section through an example of an autoinjector device 1 with the needle guard 46 pressed against a trigger site 48 following release of the plunger rod 36 by the trigger mechanism 40. The plunger rod 36 is driven toward the proximal end 22 of the autoinjector 1 through the barrel body 32 by a drive spring 50 of the drive mechanism 38, in this case the drive spring 50 is a coil spring.

FIG. 8 shows a cross section through an example of an autoinjector device 1 with the needle guard 46 pressed against a trigger site 48 at the end of the movement of the plunger rod 36 by the drive mechanism 38. In this example the stopper 34 has been driven to a proximal end of the barrel body 32 by the plunger rod 32 and drive spring 50. The stopper 34 has been driven against a shoulder 52.

The autoinjector 1 comprises an indicator component 42 which, in this example, has been triggered by the plunger rod 36 reaching, or approaching, its end of delivery position in which it holds the stopper against the stop 52. The indicator component 42, having been triggered has moved distally from its initial position (as shown in FIG. 4) to an indication position (as shown in this Figure and FIG. 9) to indicate a state of the autoinjector device 1. In this case the state being indicated is that the plunger rod 36 has reached, or approached, its end of delivery position.

The indicator component 42 in the indication position may be visible though a portion of the housing 18, for example an end cap 54. The movement of the indicator component 42 to the indication position and/or the impact of the indicator component 42 with the end cap 54 or other component, may create audible or tactile feedback to a user.

FIG. 9 shows a cross section through an example of an autoinjector device 1 following the end movement of the plunger rod 36. The autoinjector device 1 has been moved away from the trigger site which allows the needle guard 46 of the housing 18 to move away from the distal end 24.

FIG. 10 shows a cross section through an example of an autoinjector kit 100 comprising a cap 4, an autoinjector device 1 and a case 10. As shown the autoinjector device 1 has been recapped following use by having the cap 4 replaced on the proximal end of the housing 18. The autoinjector device 1 has also been placed into the case 10 after being used. In some examples the autoinjector device 1 may be placed into the case 10 prior to recapping. The case 10 may guide the housing 18 into a predetermined orientation and position within the case 10, and the case 10 may also guide the cap 4 onto the housing 18 to facilitate recapping.

To recap the autoinjector device 1 the reset rod 16 is inserted through the open proximal end 22 so that the end portion 28 passes through a stopper opening 56 in the stopper to contact the plunger rod 36. As the cap 4 is forced onto the housing 18 the reset rod 16 forces the plunger rod 36 towards its retracted position until the bearing element 26 contacts the stopper 34. Continuing to move the cap 4 onto the housing causes the plunger rod 36 to move towards its retracted position and the stopper 34 to move towards its distal position. The separation of the end portion 28 and the bearing element 26 along the longitudinal axis 20 may be greater than the axial extent of the stopper 34 resulting in a space being created between the stopper 34 and the plunger rod 36. In other examples there may be substantially no space between the stopper 34 and the plunger rod 36. In other examples there may be no stopper opening 56 and the end portion 28 of the reset rod 16 may contacts the stopper 34 and thereby force both the stopper 34 and plunger rod 36 towards the distal end 24. In other examples the reset rod 16 may not be integrated with a cap 4 so a reset rod 16 and separate cap 4 may be provided or a cap may not be provided.

During the recapping, in this example, the cap 4 contacts the needle guard 46 and forces it towards the distal end 24. When the cap 4 engages with the housing 18 the stopper 34 is in the distal position and the plunger rod 36 is in the retracted position.

In the distal position the stopper 34 may be held in place by friction forces acting between the stopper 34 and the barrel body. In the retracted position the plunger rod 36 may again be retained so that when the reset rod 16 is removed the plunger rod 36 is retained against a driving force provided by the drive mechanism 38. In this way, using the reset rod 16 the plunger rod 36, stopper 34 and drive mechanism 38 are reset.

FIG. 10 shows the autoinjector device 1 in the case 10. The case 10 comprises a reset projection 58 at a closed end 60 of the case 10. In this example the reset access opening 62 is a slot in the end cap 54 and the reset projection 58 is a wall extending from an inner wall of the case 10 along the longitudinal axis 20, but other geometries are possible.

As the autoinjector device 1 is inserted into the case 10 the reset projection 58 passes through a reset access opening 62 in a distal end 24 of the longitudinal housing 18 and contacts the indicator component 42. Following the initial contact between the indicator component 42 and the reset projection, as the autoinjector device 1 is inserted further into the case 10 the reset projection 58 passes further through a reset access opening 62 and forces the indicator component 42 from the indication position towards the initial position. When the autoinjector device 1 is fully inserted into the case 10 the indicator component 42 has returned to its initial position and, in this was the indicator component 42 is reset.

During the resetting of the plunger rod 36, stopper 34 and drive mechanism 38 and/or the resetting of the indicator component 42 the trigger mechanism 38 is reset. In this example putting the cap 4 back onto the autoinjector 1 and placing the autoinjector 1 in the case 10 resets the autoinjector 1 for future use.

FIG. 11 shows cross section through a different example of an autoinjector device 101 with a cap 4 removed. The autoinjector device 101 is similar to the autoinjector 1 of FIG. 8, but the drive mechanism 38 comprises an integrated plunger rod 136 and drive spring 150 in the form of a gas spring 64. In this example the stopper 134 does not include a stopper opening through the stopper 134.

If a reset rod 16 is used to reset the gas spring 64 the reset rod 16 may not pass through the stopper 134, but instead may contacts the stopper 134 and, by forcing the stopper 134 back to the distal position the gas spring 64 is also reset to its retracted position. In this example, with the stopper 134 in the distal position and the plunger rod 136 in the retracted positon there is no gap between the stopper 134 and the plunger rod 136.

In other examples a gap may be created between a stopper with a though hole and plunger rod 136 as described above. The drive spring 150 may comprise a gas spring 64 and an initial spring which, when the drive mechanism is triggered, drives the gas spring 64 to the stopper to recreate an initial impact of the plunger rod which may occur in a delivery device and the gas spring 64 can then drive the stopper at a controlled rate.

FIG. 12 shows a cross section through an example of a barrel body 32. The barrel body 32 includes a stopper 34 therein. The barrel body 32 has a proximal end 66 and a distal end 68. The barrel body 32 defines a channel 70, in this case a cylindrical channel, through which the stopper 34, in this case a cylindrical stopper, can slide. The resistance to movement of the stopper 34 a determined by the interaction between the stopper 34 and the barrel body 32, for example a friction force.

The friction force between the stopper 34 and the barrel body 32 may be determined by the materials from which the stopper 34 and the barrel body 32 are constructed and/or the surface finish of any contact surfaces. The friction force between the stopper 34 and the barrel body 32 may be modified by including coatings on one or more of the stopper 34 and the barrel body 32. The stopper 34 may made of an elastically deformable material such as a rubber, silicone or elastomeric material and the friction force between the stopper 34 and the barrel body 32 may be modified by changing the degree to which the stopper 34 must be deformed, or squashed, to fit within the barrel body 32.

In this case increasing the diameter of the stopper and/or reducing the diameter of the channel 70 will increase the deformation of the stopper 34 within the barrel body 32. The increased deformation may increase the force between the stopper 34 and the barrel body 32 and this may increase the resistance to movement of the stopper 34 relative to the barrel body 32. In some examples, instead of a linear transition between diameters at different sections of the barrel, the transition may be stepped. This be of use in the initial few mm of travel to recreate “headspace compression” within a prefilled syringe.

The channel 70 of the barrel body 32 may have a substantially constant diameter and this may result in a substantially constant force resisting movement of the stopper 34 within the barrel body 32. The diameter of the channel 70 may vary along the channel 70 between the distal end 68 and the proximal end 66 to vary the force required to move the stopper. The diameter of the channel 70 may increase from the distal end 68 to the proximal end 66 so that the force required to move the stopper within the barrel body 32 reduces as the stopper 34 moves to the distal end. The variation in the diameter of the channel 70 from the distal end 68 to the proximal end 66 may occur over the entire length of the channel, or over a portion of the channel. The variation in the diameter of the channel 70 may be an increase in diameter or a decrease in diameter. The result of the change in diameter along the channel 70 may result in at least a portion, or all, of the channel having a conical shape. The conical shape may have a draft angle of the channel 70 relative to the longitudinal axis of between 0.03° to 0.5° or 0.05° to 0.3°.

In this example of the barrel body 70 the barrel body includes a guide track 82. The guide track 82 may be recessed into the barrel body 70 or project therefrom and may engage with a corresponding ridge or recess on the stopper 34 and/or on the plunger rod 36 to control rotation of the stopper 34 and/or plunger rod 36. In some examples there may be no guide track 82 and in other examples there may be more than one guide track 82.

FIGS. 13 and 14 show a view and a cross section of an example of a stopper, for example the stopper 34 of FIG. 4. The stopper 34 of this example is substantially circular in cross section perpendicular to the longitudinal axis, but other shapes could be used. The stopper 34 of this example has four circumferential ribs 72 which have a similar size and shape, but other examples of stoppers may have more, fewer, or no, ribs 72. In some examples some or all of the ribs 72 may differ in size or shape when compared to others of the ribs 72.

FIGS. 15 and 16 show a view and a cross section of an example of a stopper, for example the stopper 134 of FIG. 11. The stopper 134 of this example is substantially circular in cross section perpendicular to the longitudinal axis and includes longitudinal ridges 74 extending along an exterior of the stopper 134 at positions 180° offset from one another around the circumference of the stopper 134. One or more such ridges 74 may be included in any example of a stopper 34. The stopper 134 of this example has four circumferential ribs 72 which have a similar size and shape, but other examples of stoppers may have more, fewer, or no, ribs 72. In some examples some or all of the ribs 72 may differ in size or shape when compared to others of the ribs 72.

The stopper 134 does not include a stopper opening 56 but includes a dividing wall 76 which separates proximal and distal depressions 78,80. The proximal depression 78 is sized to accept an end portion 28 of a reset rod 16. The distal depression 80 is sized to accept an end of the plunger rod 16. In other examples of a stopper 34 the stopper 35 may not include one or both of the depressions proximal and distal depressions 78,80, for example the stopper may have end walls which define a hollow interior. The end walls may include one or more openings. In other examples the stopper 34 may have a substantially solid construction.

The one or more ridges 74 on the stopper 134 may engage with a recessed guide track 82 in a barrel body 70 to control rotation of the stopper 134 as discussed above.

FIG. 17 shows an example of an endcap 54 and shows the reset access opening 62 which, in this example, is a slot in the end cap 54 through which a reset projection 58, for example a wall extending from an inner wall of a case 10 can extend and contact an indicator component 42.

FIG. 18 shows an example of a reset rod 16. In this example the reset rod 16 includes an elongate narrow end portion 28 for passing through a stopper opening 56 and engaging a plunger rod 34 as described in connection with FIG. 4. The reset rod 16 and a wider bearing element 26 for contacting a stopper as described in connection with FIG. 4. In this example the bearing element 26 is integrated into the reset rod, but may be a separate element connected, to the reset rod 16. The bearing element 26 may be connected in any suitable manner, for example by friction fit or by adhesive.

FIG. 19 shows an example of a reset rod 116. In this example the end portion 128 contacts the stopper 134 directly. In this example the end portion 128 is sized to fit within a proximal depression 78 in the stopper 134 so that the stopper 134 guides the position of the reset rod 116, in this example keeping it central within the barrel bore 32. Controlling the position of the reset rod 116 relative to the barrel body 32 may prevent the reset rod 116 from contacting the barrel body 32 and this may prevent or reduce scratches or other wear which may shorten the useful lifespan of the device.

FIG. 20 is an exploded view of an example autoinjector device 1 with a cap 4. The autoinjector device 1 comprises a longitudinal housing 18 which has an endcap 54. Within the longitudinal housing 18 there is a barrel body 32 which contains a stopper 34. The longitudinal housing 18 also contains a drive mechanism 38 and a trigger mechanism 40. Coupled to the drive mechanism 38 and trigger mechanism 40 of this example is an electronic module 84 including a battery 83, for example a button battery. A label 86 is included to wrap around portions of the longitudinal housing 18.

FIG. 21 is an exploded view of a cap 4 and proximal components of an example autoinjector device 1. The cap 4 comprises a reset rod 16 which includes a bearing element 26. The reset rod 16 is formed into a cap end 87 which couples to a cap body 88.

In this example the housing 18 includes a needle guard 46. The example needle guard 46 includes a front element 90 which defines the open proximal end 22 of the housing 18. In other examples the front element 90 may be integral with the needle guard 46. The needle guard 46 of this example includes legs 92 which, when assembled connect the needle guard 46 to the trigger mechanism 40.

The housing 18 also comprises a battery opening, for example a slot 85 through which a battery 83 can be inserted into, or removed from, the electronics module 84. A battery opening may be arranged on both sides of the housing 18. In some examples a battery slot 85 may be provided on one side of the housing 18 and a smaller battery opening on the opposite side though which a tool can be inserted to push the battery 83 out of the battery slot 85. In some examples the battery 83 may be a rechargeable battery. The rechargeable battery may be recharged by removing the battery from the autoinjector device and using a separate recharger. In some embodiments the rechargeable battery may be recharged while still within the autoinjector device, for example by wireless or wired charging, and recharging may take place while the autoinjector device is within the case.

FIG. 22 is an exploded view of an example drive mechanism 38, plunger rod 36, trigger mechanism 40 and housing endcap 54 of an example autoinjector device 1. In this case the endcap 54 includes an inner endcap component 55 fitted within the endcap 54. The endcap 54 may be transparent and the inclusion of an inner endcap component 55 may help users to avoid confusion regarding which end of the autoinjector device is the proximal end and which is the distal end.

The drive mechanism 38 comprises drive spring 50 and a guide pin 94 which helps to prevent deformation of the drive spring 50. The trigger mechanism 40 of this example comprises a plurality of latch components 96 operable to retain or release the plunger rod 36 and initiate a transition of the drive mechanism 38 from a primed state to a fired state as discussed previously.

The indicator component 42 of this example comprises two components coupled together and is biased toward the indication position by an indicator spring 98. In other examples the indicator component may be a single component, or may comprise more than two components.

FIG. 23 is an exploded view of another example drive mechanism 138, plunger rod 136, trigger mechanism 40 and housing endcap 54 of an example autoinjector device 1.

The drive mechanism 138 comprises gas spring 150 which, in this example, is integral with the plunger rod 136. The trigger mechanism 40 of this example comprises a plurality of latch components 96 operable to retain or release the plunger rod 136 and initiate a transition of the drive mechanism 138 from a primed state to a fired state as discussed previously.

FIG. 24 is an exploded view of an example of a barrel body 70, stopper 134 and plunger rod 236. The example barrel body 70 includes two longitudinal guide channels 82 with which ridges 74 of the stopper 134 engage to control rotation of the stopper 134 as it moves along the barrel body 70. The example plunger rod 236 includes a stopper contact portion 238 which includes radial projections 240 which engage with the guide channels 82 to control rotation of the plunger rod 236 as it moves along the barrel body 70.

1. An autoinjector kit, the autoinjector kit comprising an autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position;
a trigger mechanism operable to release the plunger rod and initiate a transition of the drive mechanism from the primed state to the fired state to thereby cause the plunger rod to drive the stopper from the distal position to the proximal position;
an indicator component which, when triggered, moves distally from an initial position to an indication position to indicate a state of the autoinjector device;
wherein the autoinjector kit comprises an indicator reset and the housing of the autoinjector device comprises a reset access opening in the distal end of the longitudinal housing, the indicator reset comprising a base and a reset projection, the reset projection insertable through the reset access opening to contact the indicator component and force the indicator component into the initial position.

2. The autoinjector kit as claimed in claim 1, in which the reset access opening is an elongate slot extending from the distal end of the housing towards the proximal end, and the reset projection comprises an elongate wall.

3. The autoinjector kit as claimed in claim 1, in which the base of the indicator reset comprises a case into which the housing can be fitted.

4. The autoinjector kit as claimed in claim 3, in which, when the housing is fitted into the case the housing is releasably secured therein.

5. The autoinjector kit as claimed in claim 4, in which, when the housing is releasably secured in the case by releasable clips.

6. The autoinjector kit as claimed in claim 3, in which, when the housing is fitted into the case, the reset projection extends through the reset access opening and forces the indicator component into the initial position.

7. The autoinjector kit as claimed in claim 1,
wherein the stopper comprises a stopper opening extending through the stopper along the longitudinal axis and the autoinjector kit further comprises a reset rod, the reset rod having a first end that is insertable through the open proximal end of the housing to contact the plunger rod through the stopper opening and force the plunger rod into the retracted position and thereby transition the drive mechanism to its primed state, the reset rod having a second end opposite the first that remains outside the longitudinal housing as the plunger rod is moved to the retracted position.

8. The autoinjector kit as claimed in claim 7, in which the reset rod comprises a protrusion offset from the first end along the reset rod, the protrusion sized to contact the stopper and move the stopper to the distal position as the plunger rod is moved into the retracted position.

9. The autoinjector kit as claimed in claim 8, in which the protrusion comprises a circumferential shoulder.

10. The autoinjector kit as claimed in claim 8, in which the offset between the first end and the protrusion is greater than an extent of the stopper along the longitudinal axis to create a space between the plunger rod in the retracted position and the stopper in the distal position.

11. The autoinjector kit as claimed in claim 8, in which the second end of the reset rod comprises a cap mountable to the proximal end of the housing.

12. An autoinjector device, the autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position; and
a trigger mechanism operable to release the plunger rod and initiate a transition of the drive mechanism from the primed state to the fired state to thereby cause the plunger rod to drive the stopper from the distal position to the proximal position;
wherein the autoinjector device comprises a guide track to control rotation of the plunger rod about the longitudinal axis during operation.

13. The autoinjector device as claimed in claim 1, in which the plunger rod is secured to the stopper, the stopper comprising an axial ridge on an outer surface which engages with an axial slot in the barrel body to control rotation of the plunger rod about the longitudinal axis during operation.

14. The autoinjector kit as claimed in claim 1,
the autoinjector kit further comprising a reset rod, the reset rod having a first end that is insertable through the open proximal end of the housing to apply a reset force to the plunger rod to move it into the retracted position and thereby transition the drive mechanism to its primed state, the rod having a second end opposite the first that remains outside the longitudinal housing as the plunger rod is moved to the retracted position;
wherein the maximum reset force required to move the plunger rod into the retracted position is below 10 N.

15. The autoinjector device as claimed in claim 12,
wherein an internal dimension of the barrel body perpendicular to the longitudinal axis increases towards the proximal end of the autoinjector device such that compression of the stopper is reduced as the stopper is driven by the plunger rod towards the proximal end.

16. The autoinjector device as claimed in claim 15, in which the internal dimension is an internal diameter of the barrel body.

17. A method of resetting an indicator component in an autoinjector device, the autoinjector device comprising:
a longitudinal housing extending along a longitudinal axis and having an open proximal end to contact a trigger site, a distal end opposite to the proximal end and a hollow interior;
a barrel body mounted inside the housing;
a stopper slidably mounted within the barrel body and movable from a distal position to a proximal position by a plunger rod;
a drive mechanism connected to the plunger rod, the drive mechanism having a primed state in which the plunger rod is held against a driving force in a retracted position between the distal end and the stopper in the distal position and a fired state in which the plunger rod extends through at least a portion of the barrel body and contacts the stopper in the proximal position;
a trigger mechanism operable to release the plunger rod and initiate a transition of the drive mechanism from the primed state to the fired state to thereby cause the plunger rod to drive the stopper from the distal position to the proximal position; and
an indicator component which, when triggered, moves distally from an initial position to an indication position to indicate a state of the autoinjector device;
wherein the method comprises:
inserting a reset projection of an indicator reset though a reset access opening in the distal end of the longitudinal housing of the housing of the autoinjector device to contact the indicator component and using reset projection to force the indicator component into the initial position.

18. The method as claimed in claim 17, in which the reset projection is provided in a case into which the autoinjector device can be fitted and inserting the autoinjector into the case causes the reset projection to pass though the reset access opening to contact the indicator component force the indicator component into the initial position.

19. The method as claimed in claim 17,
wherein the method further comprises:
inserting a first end of a reset rod through the open proximal end of the housing to contact the plunger rod through a stopper opening, the stopper opening extending through the stopper along the longitudinal axis; and
using the reset rod to force the plunger rod into the retracted position and thereby transitioning the drive mechanism to its primed state, a second end of the reset rod opposite the first end remains outside the longitudinal housing as the plunger rod is moved to the retracted position.

20. The method as claimed in claim 19, in which the second end of the reset rod comprises a cap for closing or covering the open proximal end of the housing of the autoinjector device, and inserting the first end of a reset rod through the open proximal end of the housing and engaging the cap with the open proximal end of the housing causes the plunger rod to move into the retracted position and thereby transition the drive mechanism to its primed state.
